(19) United States
(12) Patent Application Publication
(10) Pub. No.: US 2011/0124078 A1
(43) Pub. Date: May 26, 2011

(54) SCALABLE CELL CULTURE BIOREACTOR AND CELL CULTURE PROCESS

(75) Inventors: Wade Edwards, Cape Town (ZA); Sheena Janet Fraser, Cape Town (ZA)

(73) Assignee: SYNEXA LIFE SCIENCES (PROPRIETARY) LIMITED, Cape Town (ZA)

(21) Appl. No.: 12/991,884

(22) PCT Filed: May 11, 2009
(86) PCT No.: PCT/IB2009/051927

§ 371 Date: Jan. 19, 2011

(30) Foreign Application Priority Data
May 9, 2008 (GB) GB 0808373.5

(51) Int. Cl. C12N 11/00; C12N 5/00; C12N 1/00; C12M 1/00

(52) U.S. Cl. 435/174; 435/325; 435/243; 435/283.1; 435/289.1

(57) ABSTRACT

The present invention relates to a scaleable bioreactor comprising at least one cassette comprising a manifold and an arrangement of hollow fibre membranes; an upper headplate; and a lower headplate, wherein the cassette(s) are modular components adapted to co-operate with each other and the headplates to define an internal extracapillary culture space (ECS) and wherein the arrangement of hollow fibre membranes includes a discrete inlet and outlet. The invention further relates to a kit for such a bioreactor, a cassette for use in such a bioreactor and a process for exploiting the metabolism of cells and/or microorganisms, the process including the step of utilising a bioreactor according to the invention.

### INTRODUCTION

This invention relates to a scalable capillary (hollow fiber) membrane bioreactor and the use thereof for culturing and maintaining cells and/or microorganisms for exploiting the metabolism of these cells for the production of biological products or biotransformations, with increased process flexibility.

### BACKGROUND OF THE INVENTION

Capillary membrane, or hollow fiber reactors (HFRs) have been used extensively in upstream and downstream filtration and purification processes; as microbial or enzyme immobilised bioreactors in waste water treatment; and as cell or tissue culture systems.

Most cell culture HFR designs use axial flow, where separate fluid streams flow across the membrane surface on either side of the membrane (Chresand et al, 1987). Scale-up of these modules is undertaken by increasing the number of membranes within the hollow fiber bundle contained within a cylindrical manifold, thereby increasing the active membrane surface area. Material or nutrient exchange across the membrane surface from one fluid stream to the other occurs by diffusion. This results in axial and radial nutrient gradients across the fibre bundle which are perceived to create heterogeneities that adversely affect cell growth, bioprocess stability and productivity. These heterogeneities are amplified as the HFR is increased in size.

Cross-flow (tangential) and dead-end (flow-through) filtration in cell culture HFRs have been used to improve material exchange between two separate fluid streams, where materials were transported across the membrane surface by convective flow rather than diffusion. These two fluid delivery means have been applied to perfusion (U.S. Pat. No. 4,804,628) and immobilised (WO2007004170A2) cell culture systems, respectively. In each case enhanced delivery of nutrients to cells and the removal of metabolic waste and/or biological products of interest from the cell culturing space is observed. Nevertheless, axial gradients and heterogeneities within the hollow fiber bundle are still observed, and limit the scale of the HFR design.

Transverse-flow modules were originally developed for filtration, pervaporation and waste water treatment processes, to limit axial gradients and heterogeneities, allowing for a well-defined hydrodynamic feed and permeate flow along the membrane surface, thereby controlling concentration polarisation and optimising the concentration base driving force across the membrane (Futselaar, 1993). In these modules, capillary membranes were positioned perpendicular to the direction of the feed flow. Regular arrangement of membranes prevented flow misdistribution, while the transverse orientation increased mass transfer coefficients significantly (Futselaar, 1993). This uniform, 2-D distribution of membranes allowed for improved scalability and several designs have been described (Zhang et al 2003, Smart et al 1998, Vladisavljevic 1999). In each case the fluid path through the membranes is fixed, with all membranes orientated in the same direction manifolded into a single fluid inlet and outlet channels (FIGS. 1A-C).

U.S. Pat. No. 5,516,691 describes a modified transverse flow module, in which the membranes are distributed in a 3-D arrangement (FIG. 1D), that is used as a cell culture apparatus for culturing and maintaining cells and using their metabolisms (metabolic products). This bioreactor design encompasses the principles of improved mass transfer through cross-flow and/or dead-end filtration, and improved substrate and metabolite exchange and controllability through its 3-D network of fibres, thereby facilitating consistent fluid delivery throughout the cell culturing space and resulting in improved metabolism and cell viability. This 3-D arrangement also facilitates at least three independent membrane systems, each comprised of a plurality of membranes. The first membrane system is used for medium inflow, the second for the supply of O₂ or removal of CO₂, while the third is used for medium outflow. The inner space between the membrane systems and the outer manifold is used as the cell culturing space. This design is the first application of multi-dimensional fiber arrangement to cell culture, and was conceptualised for tissue culture as a synthetic organ or liver assist device, where scalability was not taken into consideration. Further, each membrane layer orientated in the same direction is manifolded into a single fluid inlet and outlet channel (FIG. 1D).

### SUMMARY OF THE INVENTION

According to a first aspect of the invention, there is provided a scaleable bioreactor comprising:
at least one cassette comprising a manifold and an arrangement of hollow fibre membranes;
an upper headplate; and
a lower headplate,
   wherein the cassette(s) are modular components adapted to co-operate with each other and the headplates to define an internal extracapillary culture space (ECS) and wherein the arrangement of hollow fibre membranes includes a discrete inlet and outlet.

Preferably the inlet and outlet are in fluid communication via the hollow fibre membranes. The cassette preferably comprises an inlet and outlet reservoir located in fluid communication with the (each) manifold. A manifold is preferably located at each end of the membranes and each reservoir is in fluid communication with the respective inlet or outlet.

Each cassette preferably includes only the discrete inlet and outlets in fluid communication with the reservoirs, i.e there is no fluid communication with the ECS through the cassette.

The hollow fibre membranes may be arranged in a row of substantially parallel membranes. Each cassette preferably includes one or more of such rows.

Where each cassette includes more than one such layer (row), each layer of hollow fibre membranes is preferably arranged perpendicular to the layer above and/or below it within either a horizontal or a vertical orientation. More preferably each layer of hollow fibre membranes is offset against the layer of membranes above and/or below it.

Preferably the bioreactor comprises two or more cassettes. Each cassette may include one hollow fibre membrane layer (row) per functionality.

The headplates preferably include an opening (ports) into the ECS so that fluid may be introduced into, drain from and/or be allowed to flow freely through the ECS. Headplates may be adapted to include at least one fitting or port enabling the insertion of probes or sensors into the ECS compartment, so that the composition of the fluid within the ECS may be monitored and/or controlled. Each opening (port) is preferably closable.

Preferably such ports in fluid communication with the ECS are only located on/in the headplates and not in a cassette.

According to a second aspect of the invention, there is provided a cassette comprising a manifold adapted to receive an arrangement of hollow fibre membranes, the cassette further comprising discrete inlet and outlets for each arrangement of hollow fibre membranes and wherein the cassette is adapted to engage with another cassette and/or upper and lower headplates to define an internal ECS.

According to a third aspect of the invention, there is provided a kit for a bioreactor comprising:
a manifold adapted to receive an arrangement of hollow fibre membranes; and
upper and lower headplates.

Preferably the kit includes a cassette adapted to receive or including the manifold, the cassette being adapted to co-operate with another cassette and/or the upper and lower headplates. The kit may also include at least one hollow fibre membrane, optionally fitted in the manifold.

The hollow fibre membranes are preferably axially elongated hollow tubes, shaped not unlike a drinking straw. The hollow fibre membranes are preferably selectively permeable and may be ceramic membranes.

The manifold may be included in a frame comprised of two (or more) opposed manifolds adapted to receive the hollow fibre membranes, the manifolds being joined to each other to form a frame. Each manifold may include apertures adapted to receive a hollow fibre membrane. Each cassette preferably includes (either an inlet or outlet) reservoir in fluid communication with the manifolds (apertures) and an inlet or outlet, also in fluid communication with the reservoir.

The reservoirs may be formed by cooperation of the frame with end plates.

The cassette, frame, manifold, end plates and headplates may be constructed from stainless steel or any suitable form of polymer/plastic.

In this specification, a description of each component of the bioreactor, for example, cassette, manifold, frame and/or hollow fibre membranes applies to all aspects of the invention.

According to a fourth aspect of the invention, there is provided a process for exploiting the metabolism of cells and/or microorganisms, the process including the step of utilising a bioreactor according to the invention.

The process may include inoculation of a bioreactor as hereinbefore described with the cells and/or microorganisms into the ECS. Preferably the cells and/or microorganisms are immobilised on the outer surface of the hollow fibre membranes and/or the cells and/or microorganisms are suspended in the ECS. It will be appreciated that the ECS may contain a gas and/or a liquid.

The cells may be selected from mammalian and insect cell lines and the microorganisms may be selected from bacteria, yeasts and fungi. The mammalian and insect cell lines are preferably, but not limited to, anchorage dependent types, and the microorganisms are preferably non-filamentous.

Anchorage dependent cell lines are typically selected from normal diploid cell strains, such as human embroyonic lung, human foreskin, human embroyonic kidney, chicken, rabbit, mouse and rat embryo fibroblasts, chimpanzee liver fibroblasts, rat glial cells, feline lung fibroblasts and secondary monkey kidney cells; primary cells such as monkey, dog and rabbit kidney cells, mouse macrophages, rat pancreas cells, rat hepatocytes, chicken embryo fibroblasts, rat pituitary cells and amniotic fluid cells; established and transformed cell lines, such as mouse fibroblasts, normal rat kidney, Chinese hamster ovary and lung, baby hamster kidney, chimpanzee embryo lung, African green monkey kidney, mouse L cells, HeLa, mouse macrophage cell line, transformed dog kidney, sarcoma virus transformed rat kidney and mouse fibroblasts, human glioma, human osteosarcoma, Madin-Darby canine kidney, KB cells, rhesus monkey kidney, McCoy cells, human thyroind carcinoma, human rhabdomyosarcoma, rat muscle derived fibroblasts and rabbit cornea cells.

Microrganisms are typically selected from normal or transformed Bacillus subtilis, Candida spp., Escherichia coli, Hansenula spp., Kluyveromyces lactis, Lactococcus spp., Lactobacillus spp., Staphylococcus spp, Pichia spp. Saccharomyces cerevisiae, Schizosaccharomyces pombe and Yarrowia lipolytica.

The process may include fluid flow through the inlet system of the arrangement of hollow fibre membranes by standard cross-flow operation, or by dead-end operation.

The process may include a control for modulating fluid delivery through the hollow fibre membranes. Preferably the control is a specific integrated control which may be a pressure control.

The process preferably includes the supply and/or removal of substances including nutrients, gases, such as oxygen and carbon dioxide, buffering agents (acidic or basic solutions), hormones, growth regulating or metabolism altering compounds, spent medium, metabolic waste, products of interest, and heat exchanging material. The substances may be supplied to the cells and/or microorganisms via the inlet or outlet systems of the cassettes (i.e. through the hollow fibre membranes) and/or through the openings (ports) in the upper and lower headplates.

The process may include fluid flow through the ECS and the fluid flow may further be re-circulated or may be by dead end mode. Fluid flow through the ECS may be operated under pressure. It will be appreciated that fluid flow through the ECS is preferably by transverse flow effected from the lower headplate to the upper headplate or vice versa.

The following references are included by reference herein:

1. Chresand J. J., Gillier R. J. and Dale B. E. (1987) Optimisation fibre spacing in a hollow fibre bioreactor. Biotechnol. Bioeng. 32:983-982.

2. Cracauer, R. F., Walker R. D., and Gruenberg M. (1998) Hollow fiber cell culture device and method of operation. U.S. Pat. No. 4,804,628.

3. Fraser, S. J., Edwards, W. and Leukes, W. D. (2007) Production of secondary metabolites and recombinant proteins. Synexa Life Sciences (PTY.) LTD. WO2007004170A2.

4. Futselaar H. (1993) The transverse-flow module construction, performance and application. PHD Thesis. University of Trente, Netherlands.

5. Gerlach J. (1996) Module for culturing and using metabolisms and/or for maintaining microorganisms. U.S. Pat. No. 5,516,691.

6. Smart J., Starov V. M., Schucker R. C. and Lloyd D. R. (1998) Pervaporative extraction of volatile organic compounds from aqueous systems with use of a tubular transverse flow module. J Membr Sci 143: 159-179.

7. Vladisavljevic G. T. and Mitrovic M. V. (1999) Pressure drops and hydraulic resistances in a three-phase hollow fiber membrane contactor with frame elements. Chem Eng Processing 40: 3-11.

8. Zhang S., van Houten R., Eikelboom D. H., Doddema H., Jiang Z., Fan Y. and Wang J. (2003) Bioresource Technol 185-192.

### BRIEF DESCRIPTION OF THE DRAWINGS

The invention will now be described in more detail, by way of example only, with reference to the following non-limiting drawings.

FIG. 1 Shows various prior art arrangements.

FIG. 2 Shows a (A) cross section, (B) top or bottom view and (C, D) perspective views of a complete scalable bioreactor according to the invention.

FIG. 3 Shows an exploded view of a scalable bioreactor according to the invention.

FIG. 4 Shows a schematic of a process example.

FIG. 5 Shows a bioreactor setup used in Example 1.

FIG. 6 Shows CHO-K1 process data for Example 1.

### DETAILED DESCRIPTION OF PREFERRED EMBODIMENTS

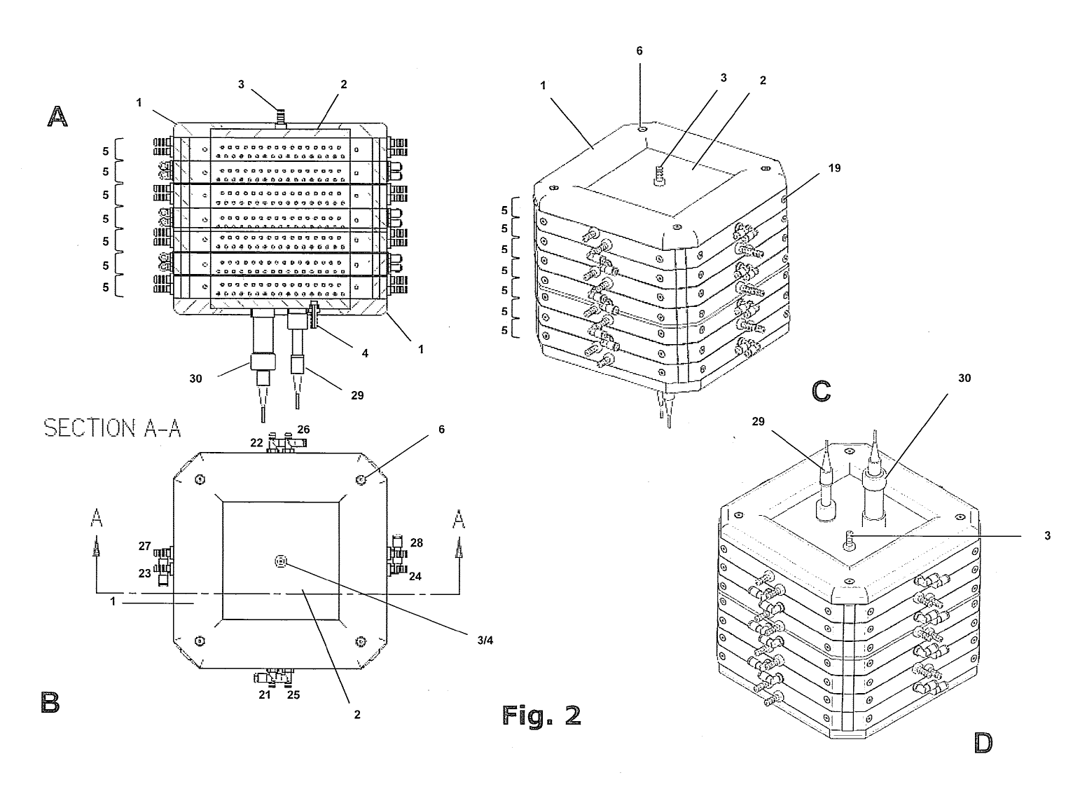

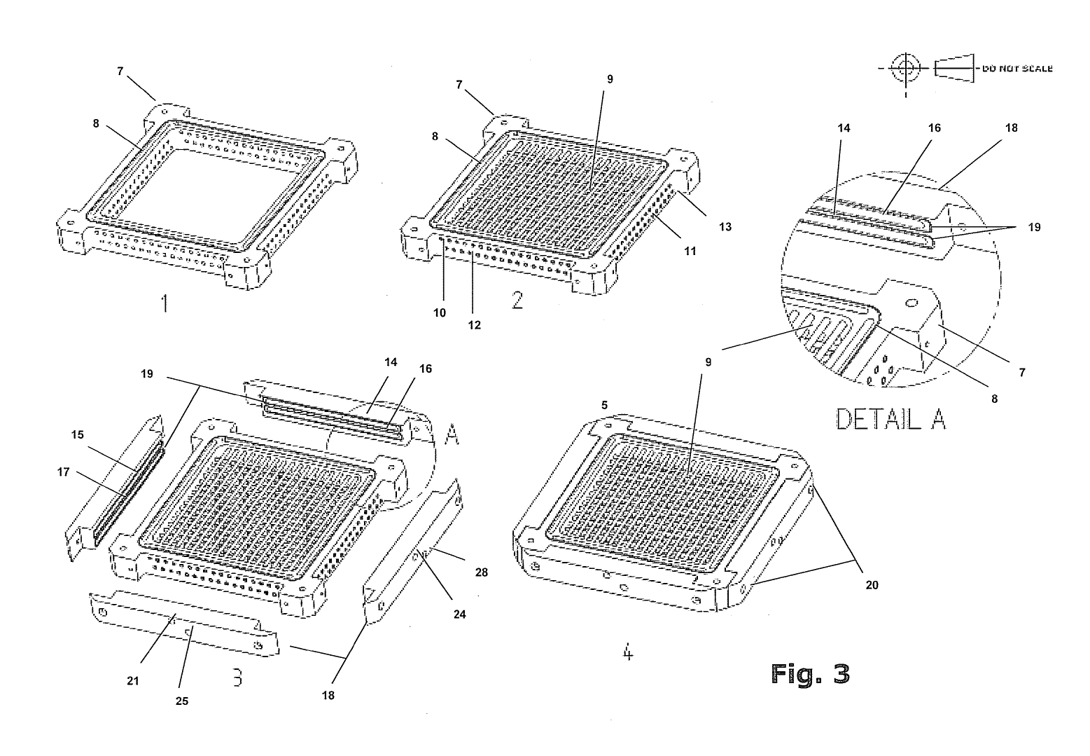

In the current design the principles of modular scale-up and process flexibility are incorporated in the design of a 2-D and 3-D network of capillary membranes. The scalable membrane bioreactor apparatus (FIGS. 2 and 3) is comprised of two headplates (1) and at least one cassette (5) secured between the two headplates, effectively defining the extracapillary space (ECS) and separating it from the intracapillary space (ICS) located within the hollow fibre membranes. FIG. 2 illustrates the complete cell culture module, showing two headplates (1) securing, by example, seven separate cassettes (5). Upper and/or lower headplates may include a central transparent viewing panel (2) that allows the conditions within the module to be observed during operation. Each headplate may also include at least one ECS port (3, 4) acting as an inlet/outlet to the ECS and at least one fitting or port enabling for the insertion of probes or sensors into the ECS compartment, by way of example fittings for pH (29) or DO probes (30). Each cassette (5) is sealed against the adjacent cassette (5) and/or headplate (1) using an O-ring, gasket or by other means (8). The cassette and headplate seals are compressed together and secured in place by way of, for example, four rods or pins (6) situated at the four corners of the stacked apparatus. Larger bioreactors may require additional pins to facilitate compression.

Each cassette (FIG. 3) is comprised of an insert frame (7) in which at least four layers of capillary membranes (9) are secured using resin, epoxy or by other means. In the preferred embodiment, the capillary membranes are ceramic, offering rigidity to the frame and resistance to structural distortion frequently observed with polymer membranes. However, it should be noted that this design is not limited to capillaries of any particular dimension, chemical properties or technical characteristics. In the preferred embodiment, each layer is comprised of a similar type of membrane, but different layers may be comprised of membranes with different chemical or technical properties, including, but not limited to, manufacturing material, pore size and distribution, coating, selectivity and/or conductivity.

The 2-D membrane array (9) ensures the uniform supply and removal of materials through the membranes, at all positions within the reactor, thereby minimising axial gradients and heterogeneities within the bioreactor. By operating the module under pressure, higher concentrations of dissolved nutrients or gas may be achieved in the cell culture fluid. Dead end flow of nutrients from one membrane system (layer or row) into the ECS (cell culture space) and out through a second membrane system may serve to concentrate and immobilise cells on the membrane surface and ensures improved mass transfer and uniform delivery of nutrients to all immobilised cells within a biofilm. Cell concentration and biofilm formation has been correlated with cell-cell communication, cell-viability and enhanced productivity.

Each successive layer of membranes are received within rows of apertures (10, 11, 12, 13) in the manifold, each layer being orientated in a perpendicular direction to the membrane layer above and below it. Furthermore, each successive membrane layer orientated in the same direction may be offset in such a way that when observed from above, the membranes are staggered to form a continuous biaxial membrane network (9), limiting misdistribution of fluids and gravity assisted sedimentation of cells within the ECS through channelling.

Each membrane layer is effectively compartmentalised from the other membrane layers by a fluid distribution manifold included on the frame (7). The open ends of each capillary membrane within a membrane layer open into and are contained by separate fluid distribution chambers (reservoirs)(14, 15). Different membrane layers and fluid distribution chambers (14, 15) within a single cassette (5) are sealed from one another by O-rings, gaskets or by other means (19). Each of four separate end plates(18) are aligned and sealed against the frame (7) and secured in place by at least two screws or pins (20) to define the fluid distribution chambers (reservoirs). Each membrane layer has in each opposed fluid distribution chamber an ICS inlet (21, 23, 25, 27) or ICS outlet (22, 24, 26, 28) which enables separate and contained fluid flow through each membrane layer.

In the preferred embodiment, at least one membrane layer is used to supply a nutrient solution to the cells in the ECS, while at least one layer is used to remove spent medium containing the product of interest and/or metabolic waste. Additional layers may be used:
to oxygenate the culture space using gas permeable membranes;
to buffer the culture space by regulating CO₂ concentrations introducing an acidic or basic solution, or scavenging organic acids from the culture space using selectively permeable membranes;
for the controlled supply of growth regulating or metabolism altering compounds for enhanced process stability and control. Including, but not limited to, specific nutrient feeds, growth-phase inhibitors or inducer molecules; or
as a heat exchanger to maintain the reactor at a constant temperature.

The cell culture space may be drained, purged or re-circulated with a nutrient solution through the inlet(s) or outlet(s) located on the headplates.

The modular assembly offers increased flexibility in scale from the same design, where once the process has been optimised with a bioreactor comprised of at least one membrane insert, additional membrane inserts may be stacked on top of one another, as required, effectively increasing the membrane surface area and reactor volume linearly. For a given process, membrane layers from different membrane inserts may be manifolded together to facilitate uniform distribution of fluid delivery and/or removal to/from all membrane inserts within the module.

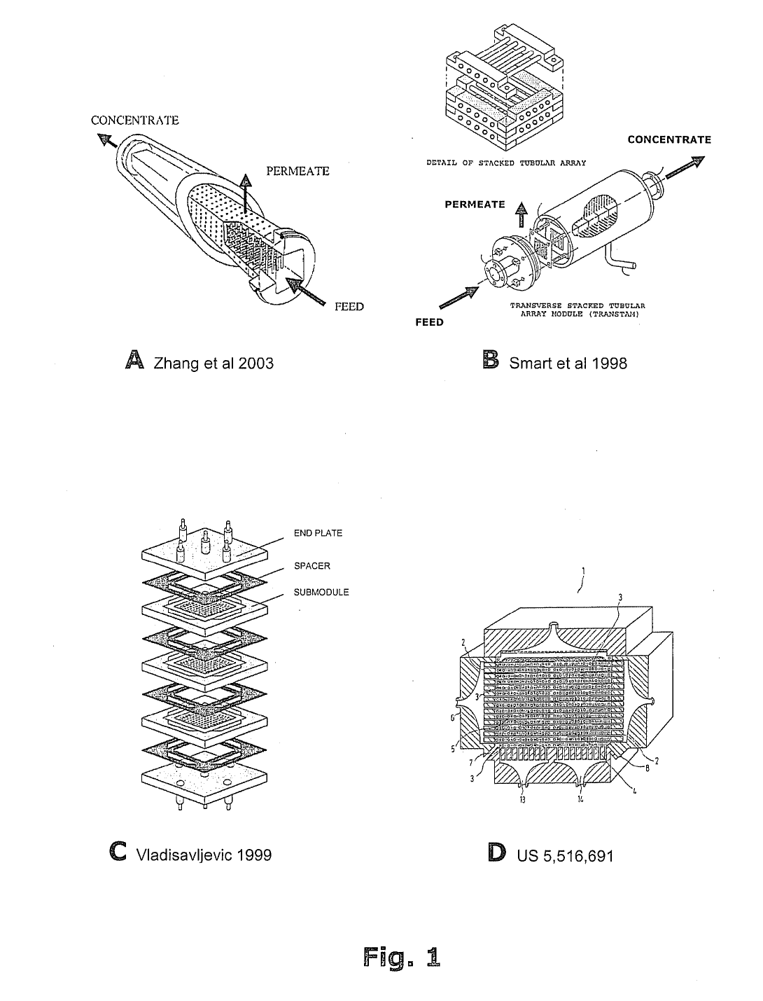

In the preferred embodiment, the bioreactor apparatus is orientated vertically and the membrane network is orientated horizontally (FIG. 2, section A-A). In this instance the ECS port (4) located on the basal headplate (1) would serve to introduce a cell inoculum and/or fluid into the ECS, while allowing air or spent medium to exit through the ECS port (3) located on the top headplate. In this way the entire ECS may be primed with fluid, preventing air-locks. This is essential for the effective operation of any probes used to monitor process conditions within ECS, including, but not limited to pH (29) or DO probes (30) Fluid flow, transverse to the membrane network, may be re-circulated through the ECS using standard cross-flow operating conditions (see FIG. 1, prior art) or the ECS outlet (3) may be closed and the fluid flow operated in dead-end mode. Either flow mode can be used to immobilise cells on the outer surface of at least one hollow fibre included in a membrane layer within a cassette (5). This is facilitated by applying pressure to the fluid in the ECS such that the liquid is forced to permeate through the wall of selectively permeable capillary membranes (9) into the ICS and exiting through at least one ICS exit port (22, 24, 26, 28), while the inoculum is retained at the membrane surface on the ECS side.

During long term operation, cell debris or materials that may accumulate within the ECS and sediment under the influence of gravity may be purged from the ECS using the ECS port (4) located on the basal headplate.

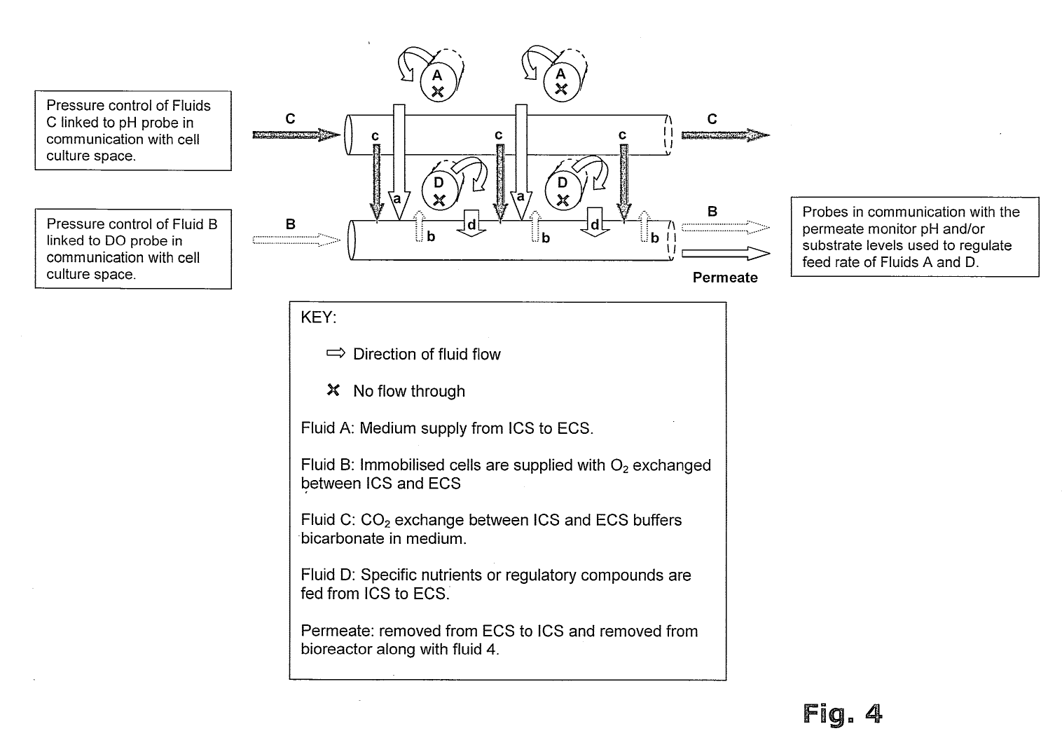

Following inoculation the ECS ports (3, 4) are sealed at either end, effectively containing the cell culture space in the ECS. By way of example (FIGS. 2, 3 and 4), nutrient solution is passed through the ICS of a first membrane layer or system (A), entering at the membrane layer ICS inlet (21), filling the inlet fluid distribution chamber (inlet reservoir), the lumen of all capillaries in the membrane layer, the fluid distribution chamber (outlet reservoir)at the outlet (14) and exiting through the ICS outlet (22) of the same membrane layer. During operation ICS outlet (22) of this membrane layer is blocked and medium delivery is operated in dead-end mode. Fresh nutrient solution is filtered from the ICS of all capillaries in this first membrane layer (a), across the membrane wall into the bioreactor ECS where fresh nutrients may be metabolised by cells. Under pressure, spent medium/permeate exits the ECS by filtering through the capillary wall into the ICS of a second membrane layer and exits the membrane layer ICS through the ICS outlet (28) of the second membrane layer. Spent medium/permeate is collected in a permeate collection vessel. In this way fresh nutrients are continuously supplied to the immobilised cells/biofilm while metabolic waste that may adversely effect cell growth is continuously removed from the cell culture space. Further, depending on the molecular characteristics of a product of interest and the physical characteristics of the capillary membranes within the second membrane layer, secreted product may be continuously produced and removed from the bioreactor along with the permeate, thereby retaining the biomass and preventing inhibition of cell growth by toxic metabolites, negative feedback control of product formation and/or enzymatic degradation of labile products.

Under continuous operation a cell culture process may require additional nutrients such as increased dissolved oxygen (DO) for high density cell growth. The lumen of the second membrane layer may be aerated (B), where humidified air is supplied at the ICS inlet of the second membrane layer (27), enter the fluid distribution chamber at the inlet (inlet reservoir), flow through the ICS of all capillaries in the membrane layer, into the fluid distribution chamber at the outlet (outlet reservoir), exiting at the ICS outlet (28) of the same membrane layer, together with the permeate. Dissolved oxygen may diffuse from the ICS, across the membrane wall towards the ECS (b) where it may be metabolised by immobilised cells/biofilm. The rate of oxygenation may be regulated by an integrated process control using DO probes e.g. by modulating the O₂ levels in the humidified air stream.

A third membrane layer (C) may be used to regulate the pH of the cell culture fluid through the controlled supply of acidic or basic solutions, carbon dioxide (CO₂) or bicarbonate. These solutes or dissolved metabolites may be supplied at the ICS inlet of a third membrane layer (23), into a fluid distribution chamber (inlet reservoir) at the ICS inlet, through the lumen of all capillaries in the membrane layer, into the fluid distribution chamber at the outlet (outlet reservoir) exiting at the ICS outlet (24) of the same membrane layer. Buffering compounds may be supplied from the ICS to the ECS across the membrane wall (c). The rate of delivery of buffering compounds across the membrane wall, from the ICS into the cell culture fluid within the ECS may be controlled by integrated process control using pH probes. e.g. integrated pressure control for modulating fluid delivery across the membrane wall into the ECS.

A fourth membrane layer may be used to feed specific growth regulating factors or other compounds (D) influencing cell metabolism and/or the production of compounds of interest. These compounds may be expensive or labile. Such compounds may be fed (D) into the cell culture space under pressure through the ICS inlet of a fourth membrane layer (25), into the inlet fluid delivery chamber (inlet reservoir), through the lumen of capillaries in the fourth membrane layer, into the fluid delivery chamber at the outlet (outlet reservoir), exiting through the ICS outlet (26) of the same membrane system. The rate of delivery of specific metabolites (d) across the membrane wall, from the ICS into the cell culture fluid within the ECS may be controlled using process specific integrated control strategies. e.g. integrated pressure control for modulating fluid delivery across the membrane wall into the ECS.

It should be noted that the number of membrane systems (layers), the composition and the sequence of materials delivered to the cell culture space may differ but the principles of improved mass transfer, independent fluid delivery and integrated process control remains the same. All compounds delivered into the ECS from the ICS of different membrane systems are transported within the ECS and through the immobilised cell layer/biofilm by convective flow. As these materials pass through the immobilised cell layer/biofilm they are metabolised by the cells, resulting in self-regulating gradients that are established across the biofilm. The nature of these gradients is fully controlled in an integrated process, and allows for consistent process conditions that may be used to regulate morphological differentiation and growth phase related metabolism(s) in a continuous process, the scale of which is defined by the number and size of the membrane inserts used.

### EXAMPLE

Cultivation of Chinese Hamster Ovary (CHO) cells immobilized within the ECS of a scalable cell culture bioreactor with single cassette.

CHO cells are routinely used in biological and medical research, including toxicity screening, nutrition and gene expression. CHO cells are the most commonly used mammalian hosts used for the production of recombinant protein therapeutics.

CHO-K1 is an anchorage-dependent cell line which typically grows as a monolayer on suitable cell culture surfaces and in our laboratory routinely grows to a cell density of approximately 1×10⁵ cells/cm² surface area in cell culture flasks.

Strain & Culture Conditions

Adherent CHO-K1 cells were routinely cultivated in T-75 Flasks using 30 ml DMEM-F12 growth medium containing 2 mM glutamine, supplemented with 5% (v/v) fetal bovine serum (FBS) and 1% (v/v) Pen/Strep. Cells were passaged every 3-4 days. T-150 Flasks containing 80 ml growth medium, were used for culture expansion. All cultivations were carried out in a CO₂-incubator at 37° C.

For inoculation, cells were suspended in 15-20 ml growth medium. In this example, approximately 2.5×10⁷ cells were used as the inoculum.

Reactor Setup

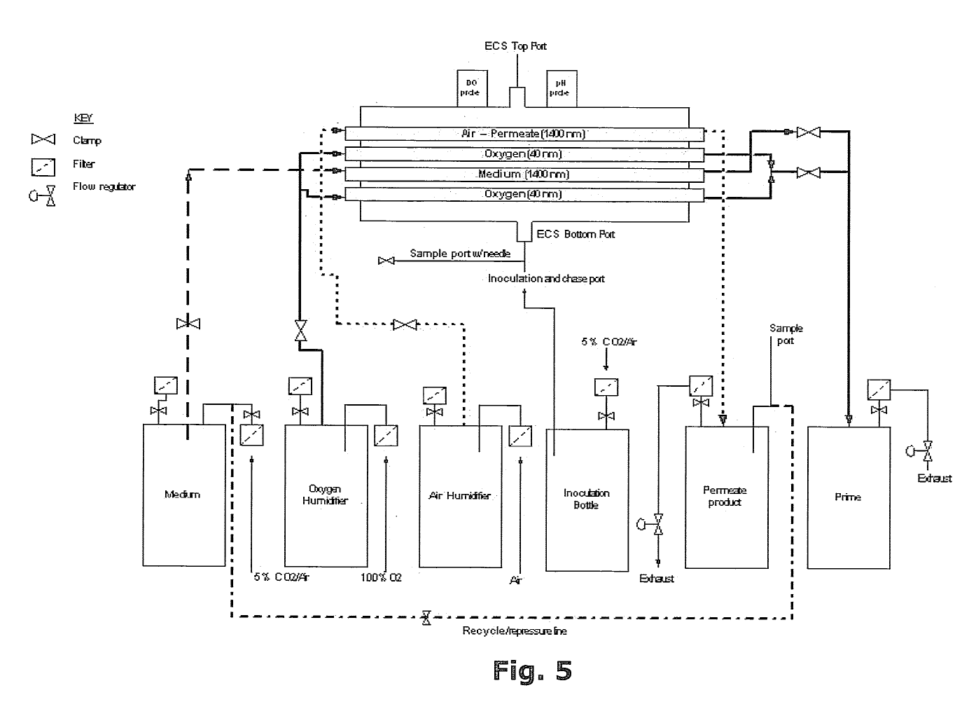

A disposable bioreactor module, comprised of a single cassette secured by headplates was used for CHO-K1 cultivation. The bioreactor design provided a total capillary surface area of 519 cm² for adherent cell growth, in a working volume (ECS volume) of 119 cm³. Bioreactor and ancillary setup is outlined in FIG. 5 and includes pH and dissolved oxygen probes integrated into one of the headplates in order to monitor process conditions within the ECS. In this example, each of four ceramic membrane layers within the cassette included membranes of the same chemical composition but different pore sizes, such that each membrane layer facilitated a different function during operation, as described below.
Layer 1: 1400 nm ceramic capillaries—spent nutrients removed through lumen, with aeration
Layer 2: 40 nm ceramic Capillaries—100% oxygen supplied through lumen
Layer 3: 1400 nm ceramic capillaries—nutrient solution supplied through lumen
Layer 4: 40 nm ceramic capillaries—100% oxygen supplied through lumen

Growth medium was supplied pneumatically using 5% carbon dioxide/compressed air such that nutrients flow from the medium supply vessel through the lumen (ICS) of all membranes positioned in layer 3. Prior to operation, air was displaced from reticulation and the membrane lumen by priming the medium from the medium supply vessel, through layer 3 ICS and into a prime collection vessel. During operation the ICS outlet of layer 3 is clamped closed and the medium supply is operated in dead-end mode such that nutrients are forced from the ICS through the capillary membrane walls and into the ECS or cell growth compartment. Spent medium is displaced from the ECS and removed via membrane layer 1.

Similarly, 100% oxygen is supplied through the ICS of capillary membranes located in layers 2 and 4. Oxygen was supplied at a pressure greater than or equal to that of the medium supply, depending on the oxygen uptake requirements of the cells growing within the cell growth compartment. Bubble-free oxygenation is primarily diffusive and is promoted by gas flow across the ICS surface. Back pressure is maintained by a flow control valve also used to maintain a specific flow rate.

The final layer has a dual function: firstly, compressed air flows from a humidification vessel through the ICS of layer 1 into a collection vessel before it is released through a flow control valve. The flow control valve maintains air flow at a specific flow rate and additionally provides backpressure required for operation; secondly, during operation spent medium is removed from the ECS, across the capillary walls and into the ICS of layer 1 at a rate equivalent to the medium supply rate. Permeate entering the lumen of membranes in layer 1 is drained into the collection vessel along with the airflow through this layer. Permeate may be sampled or removed from the collection vessel via a sampling port.

Sterilisation

The disposable bioreactor module with reticulation and sterile connectors was gamma-irradiated. Medium supply vessel, prime vessel, humidification vessels, permeate collection vessel and inoculation vessel, each with reticulation and sterile connectors were autoclaved separately and aseptically connected to the gamma-irradiated bioreactor module prior to inoculation.

Inoculation

Prior to inoculation the sterile bioreactor setup and growth medium was pre-incubated at 37° C. Growth medium was sparged and equilibrated to pH 7.2 with 5% carbon dioxide/air.

2.5×10⁷ CHO-K1 cells were inoculated directly into the bioreactor ECS. Cells were immobilized on the outer surface of capillary membranes by priming the ECS with DMEM-F12 growth medium containing 2 mM glutamine, supplemented with 10% (v/v) fetal bovine serum (FBS) and 1% (v/v) Pen/Strep. Medium flow from the inoculation vessel into the ECS was directed under pressure across membrane walls, into the capillary lumen (ICS) towards the prime vessel before being recycled into the medium supply vessel prior to operation.

Operation

Cells were cultured at 37° C. using DMEM-F12 growth medium containing 2 mM glutamine, supplemented with 10% (v/v) fetal bovine serum (FBS) and 1% (v/v) Pen/Strep, equilibrated to pH 7.2 with 5% carbon dioxide/air.

Following inoculation the bioreactor was operated with constant air flow and pressure (40 kPa) through the ICS of membrane layer 1 and with a constant 100% oxygen flow and pressure (50-70 kPa) through membrane layers 2 and 4. Oxygen pressure was adjusted at intervals in order to support increased oxygen delivery and maintain the DO concentration (10-30%) within the ECS, for enhanced cell growth and viability. Oxygen pressure was never lower than the pressure applied to the medium supply vessel, preventing spent medium flow into these membrane layers.

As the CHO cells grow, nutrients are depleted and medium pH within the ECS (cell growth environment) is altered due to waste products produced by cell metabolism. Spent medium was removed from the ECS and displaced into the ICS of membrane layer 1 as fresh medium was supplied to the ECS from the ICS of membrane layer 3. The spent medium was in turn displaced from the ICS of membrane layer 1 and transferred into a collection vessel along with the air flowing through the ICS of membrane layer 1. The rate at which fresh medium was supplied to and spent medium removed from the ECS is regulated by the pressure differential between the pneumatic pressure applied to the medium supply vessel (>40 kPa) and the constant pressure applied to the ICS of membrane layer 1 (=40 kPa). The flow rate was adjusted to exceed nutrient demand by growing cells (1-10 ml/hr).

During operation, the pH within the ECS was monitored and the medium supply was spiked with small volumes of bicarbonate at intervals, to compensate for increased waste accumulation at high cell density, and when medium feed rate was unable to buffer the cell growth environment. In this way the ECS pH was maintained above pH 6.8.

Bioreactor operation was continuous and cells were maintained for a period of 11 days before the membrane module was dissected and CHO-K1 numbers were counted using trypan blue. Spent medium was sampled from the collection vessel daily for substrate analysis.

Results & Discussion

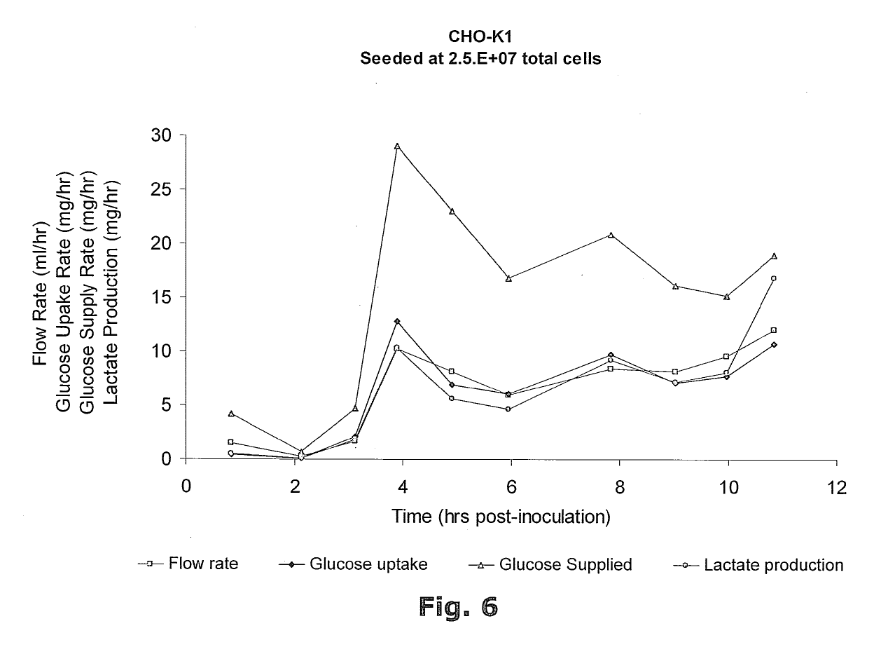

CHO-K1 cell cultivation was operated continuously for 11 days. From FIG. 6, substrate analysis showed that the nutrient supply exceeded demand at all times during the cultivation. Following inoculation the glucose uptake rate by cells was low, indicating a 1-2 day lag in growth. Following day 2 glucose uptake rate was seen to increase from 0.1 mg/hr up to a maximum of 12 mg/hr at day 4 after which the glucose uptake rate leveled off and stabilized at approximately 8 mg/hr for the remainder of the cultivation. Indicating that maximum cell yield was obtained by day 4.

Lactate production followed a similar trend to the glucose uptake rate, with levels glucose metabolized to lactate by the cells remaining below the uptake rate up until day 9 at which point the cells became stressed by low medium pH, which dropped below pH 6.8. When it became apparent that the cell metabolism was no longer optimal the growth experiment was stopped and the total cell yield determined.

A total cell yield of 1.5×10⁸ cells was obtained. Of these, 1.5×10⁷ cells were loosely adhered or suspended in the ECS, while 1.3×10⁸ adherent cells were harvested after trypsinization.

In this experiment, an adherent cell density of 2.5×10⁵ cells/cm² was obtained, comparing favorably with the maximum CHO cell densities for T175 flask (1.7×10⁵ cells/cm²) and HYPERFlask (2.2×10⁵ cells/cm²) cultivations, reported by Syzmanski et al (2008).

### REFERENCE

1. Szymanski, S. L, Huff, K. W, Patel, A. D., Murray, J. R, Feasby, J., Sharma, B. V., Denise Young, D. K., Strulovici, B., Peltier, R. R., Johnson, E. N. and Rush, A. (2008) Automated application of a novel high yield, high performance tissue culture flask. JALA vol. 13(3): 136-144.

1. A scaleable bioreactor comprising:
at least one cassette comprising a manifold and an arrangement of hollow fibre membranes;
an upper headplate: and
a lower headplate;
wherein the cassette is a modular components adapted to co-operate with each other and the upper and lower headplates to define an internal extracapillary culture space (ECS), and wherein the arrangement of hollow fibre membranes includes a discrete inlet and outlet.

2. A scaleable bioreactor according to claim 1 wherein the arrangement of hollow fibre membranes comprises an inlet reservoir and an outlet reservoir located at a manifold at each end of the membranes, each reservoir being in fluid communication with the respective inlet or outlet.

3. A scaleable bioreactor according to claim 1 wherein the hollow fibre membranes are arranged in a layer of substantially parallel membranes.

4. A scaleable bioreactor according to claim 3 wherein the cassette includes one or more of the layers.

5. A scaleable bioreactor according to claim 3 wherein the cassette includes more than one layer, each layer of hollow fibre membranes arranged perpendicular to the layer above and/or below in a horizontal or a vertical orientation.

6. A scaleable bioreactor according to claim 3 wherein the layer of hollow fibre membranes is offset against a layer of membranes disposed above and/or below.

7. A scaleable bioreactor according claim 3 wherein the cassette includes one hollow fibre membrane layer per functionality.

8. A cassette comprising
a manifold adapted to receive an arrangement of hollow fibre membranes,
discrete inlet and outlets for each arrangement of hollow fibre membranes, and
wherein the cassette is adapted to engage with another cassette and/or upper and lower headplates to define an internal ECS.

9. A kit for a bioreactor comprising a manifold adapted to receive an arrangement of hollow fibre membranes and upper and lower headplates.

10. A kit according to claim 9 including a cassette adapted to receive the manifold, the cassette being adapted to co-operate with another cassette and/or the upper and lower headplates.

11. A kit according to claim 10 including at least one hollow fibre membrane continued to be fitted in the manifold.

12. A kit according to claim 10 wherein the cassette includes two opposed manifolds adapted to receive the hollow fibre membranes, the manifolds joined to each other by side supports to form a frame.

13. A process for exploiting the metabolism of cells and/or microorganisms, the process including the step of utilising a bioreactor according to claim 1.

14. A process according to claim 13 wherein the process includes inoculation of the bioreactor with the cells and/or microorganisms into the ECS.

15. A process according to claim 13 wherein the cells and/or microorganisms are immobilised on the outer surface of the hollow fibre membranes and/or the cells and/or microorganisms are suspended in the ECS.

16. A process according to claim 13 including fluid flow through the inlet system of the arrangement of hollow fibre membranes by standard cross-flow operation, or by dead-end operation.

17. A process according to claim 13 including a control for modulating fluid delivery through the hollow fibre membranes.

18. A process according to claim 13 including the supply and/or removal of substances including nutrients, gases, such as oxygen and carbon dioxide, buffering agents (acidic or basic solutions), hormones, growth regulating or metabolism altering compounds, spent medium, metabolic waste, products of interest and heat exchanging material, wherein the substances are supplied to the bioreactorvia the inlet or outlet systems of the cassettes and/or through the openings (ports) in the upper and lower headplates.

19. A process according to claim 13 including fluid flow through the ECS wherein the fluid flow is further re-circulated or is circulated in dead end mode.
